(19) United States
(12) Patent Application Publication
(10) Pub. No.: US 2022/0251614 A1
(43) Pub. Date: Aug. 11, 2022

(54) RECOMBINANT CELL, EXTRACT, CONSUMABLE PRODUCT AND METHOD FOR PRODUCTION OF BIOACTIVE PLANT METABOLITE

(71) Applicant: Brightseed, Inc., South San Francisco, CA (US)

(72) Inventors: James Flatt, South San Francisco, CA (US); Chuan Wang, South San Francisco, CA (US); Jessica Leigh Ochoa, South San Francisco, CA (US); Cliff Rutt, South San Francisco, CA (US)

(21) Appl. No.: 17/726,926

(22) Filed: Apr. 22, 2022

Related U.S. Application Data
(63) Continuation of application No. PCT/US2020/056887, filed on Oct. 22, 2020.
(60) Provisional application No. 62/925,941, filed on Oct. 25, 2019.

(51) Int. Cl. C12P 13/02; C12N 1/18; C12N 15/81; C12N 15/52; C12P 13/22

(52) U.S. Cl. CPC C12P 13/02; C12N 1/18; C12N 15/81; C12R 2001/865; C12P 13/22; C12Y 401/01025; C12Y 203/0111; C12N 15/52

(57) ABSTRACT

Disclosed herein are recombinant host cells and methods capable of producing a tyramine containing hydroxycinnamic acid amide compounds, derivatives and extracts. Some embodiments provided include, for example, consumable products containing the tyramine containing hydroxycinnamic acid amide produced by the recombinant host cells. Some embodiments provided herein are methods for producing a tyramine containing hydroxycinnamic acid amide or hydroxycinnamic acid amide derivative.

### CROSS-REFERENCE TO RELATED APPLICATIONS

This patent application is a continuation of PCT Application No. PCT/US2020/056887, filed Oct. 22, 2020 and claims the benefit of priority to U.S. Provisional Application No. 62/925,941, filed Oct. 25, 2019. The foregoing application is fully incorporated herein by reference in their entireties for all purposes.

### BACKGROUND

N-Hydroxycinnamic acid amides (HCAAs) are synthesized by the condensation of hydroxycinnamoyl-CoA thioesters and aromatic amines. The hydroxycinnamoyl-CoA thioesters include cinnamoyl-CoA, p-coumaroyl-CoA, caffeoyl-CoA, feruloyl-CoA, and sinapoyl-CoA, and are synthesized from cinnamic acid by a series of enzymes, including cinnamate-4-hydroxylase, coumarate-3-hydroxylase, caffeic acid O-methyltransferase, ferulate-5-hydroxylase, and hydroxycinnamate:CoA ligase (Douglas (1996) Trends Plant Sci 1: 171-178).

Tyramine-derived HCAAs are commonly associated with the cell wall of tissues near pathogen-infected or wound healing regions. Moreover, feruloyltyramine and feruloyloctapamine are covalent cell wall constituents of both natural and wound periderms of potato (Solanum tuberosum) tubers, and are putative components of the aromatic domain of suberin. The deposition of HCAAs is thought to create a barrier against pathogens by reducing cell wall digestibility. HCAAs are formed by the condensation of hydroxycinnamoyl-CoA thioesters with phenylethylamines such as tyramine, or polyamines such as putrescine. The ultimate step in tyramine-derived HCAA biosynthesis is catalyzed by hydroxycinnamoyl-CoA:tyramine N-(hydroxycinnamoyl)transferase.

Plant-specific feruloyltyramine, p-coumaroyltyramine, and caffeoyltyramine have been produced in Escherichia coli by heterologous expression of two biosynthetic genes encoding p-coumarate:coenzyme A ligase and tyramine N-hydroxycinnamoyltransferase cloned from Arabidopsis thaliana and pepper, respectively (Kang, et al. (2009) Biotechnol. Lett. 31(9):1469-75). In addition, transgenic rice seeds expressing tyramine N-hydroxycinnamoyltransferase and tyrosine decarboxylase from a single self-processing polypeptide have been described (Park, et al. (2009) Biotechnol. Lett. 31(6):911-5). Further, the metabolic pathways for synthesis of N-hydroxycinnamoyl phenethylamines and tyramines were reconstructed in E. coli by expressing several genes including 4-coumarate-CoA ligase, tyramine N-hydroxycinnamoyl transferase or phenethylamine N-hydroxycinnamoyl transferase, phenylalanine decarboxylase or tyrosine decarboxylase, and tyrosine ammonia lyase and engineering the shikimate metabolic pathway to increase endogenous tyrosine concentration in E. coli (Sim, et al. (2015) Microbial Cell Fact. 14:162).

### SUMMARY OF THE DISCLOSURE

This disclosure provides a recombinant eukaryotic host cell capable of producing a tyramine containing hydroxycinnamic acid amide, wherein said recombinant host overproduces L-tyrosine or L-phenylalanine; and harbors one or more nucleic acid molecules encoding one or more enzymes of a phenylpropanoid CoA pathway for making a hydroxycinnamoyl-CoA ester; a nucleic acid molecule encoding a tyrosine decarboxylase (E.C. 4.1.1.25); and a nucleic acid molecule encoding a tyramine N-hydroxycinnamoyltransferase (E.C. 2.3.1.110). In some embodiments, the tyramine containing hydroxycinnamic acid amide is N-caffeoyltyramine, N-feruloyltyramine, 5-hydroxyferuloyltyramine, p-coumaroyltyramine, cinnamoyltyramine or sinapoyltyramine. In other embodiments, the one or more nucleic acid molecules encoding one or more enzymes of a phenylpropanoid CoA pathway for making a hydroxycinnamoyl-CoA ester include phenylalanine ammonia lyase, 4-coumarate-CoA ligase, cinnamate-4-hydroxylase, coumarate-3-hydroxylase, caffeoyl-CoA O-methyltransferase, ferulate-5-hydroxylase, caffeic acid/5-hydroxyferulic acid O-methyltransferase, tyrosine ammonia lyase, or a combination thereof. In further embodiments, the host cell overproduces S-adenosyl-methionine. A method for producing a tyramine containing hydroxycinnamic acid amide using the recombinant eukaryotic host cell, as well as an extract and consumable product containing the tyramine containing hydroxycinnamic acid amide are also provided.

### BRIEF DESCRIPTION OF THE DRAWINGS

FIG. 1 depicts a schematic pathway for the biosynthesis of tyramine containing hydroxycinnamic acid amides from hydroxycinnamoyl-CoA esters and tyramine. However, cofactors and co-substrates are not shown for clarity. Enzymes of the phenylpropanoid pathway are phenylalanine ammonia-lyase (PAL, E.C. 4.3.1.24); cinnamate-4-hydroxylase (C4H, E.C. 1.14.14.91); p-coumaroyl-CoA ligase (4CL, E.C. 6.2.1.12); coumarate-3-hydroxylase (C3H, E.C. 1.14.13.-); coumaroyl-CoA 3-hydroxylase (CCoA3H, or 5-O-(4-coumaroyl)-D-quinate 3′-monooxygenase, E.C. 1.14.14.96); caffeoyl-CoA O-methyltransferase (CCoAOMT, E.C. 2.1.1.104); ferulate-5-hydroxylase (F5H, E.C. 1.14.-.-); and caffeic acid/5-hydroxyferulic acid O-methyltransferase (COMT, E.C. 2.1.1.68). Additional enzymes in the biosynthesis of tyramine containing hydroxycinnamic acid amides include hydroxycinnamoyl CoA:tyramine hydroxycinnamoyltransferase (THT, E.C. 2.3.1.110); tyrosine ammonia lyase (TAL, E.C. 4.3.1.23), phenylalanine hydroxylase (PAH, E.C. 1.14.16.1) and tyrosine decarboxylase (TYDC, E.C. 4.1.1.25).

FIG. 2 shows a schematic representation of engineered pathways in S. cerevisiae and E. coli for overproduction of phenylalanine and/or tyrosine. Erythrose 4-phosphate (E4P), phosphoenolpyruvate (PEP), 3-deoxy-D-arabino-heptulosonic acid 7-phosphate (DAHP), 3-dehydroquinate (DHQ), 3-dehydro-shikimate (DHS), shikimate (SHIK), shikimate-3-phosphate (SHP), 5-enolpyruvylshikimate-3-phosphate (EP3P), prephenate (PPA), phenylpyruvate (PPY), para-hydroxy-phenylpyruvate (HPP), phenylacetaldehyde (PAC), para-hydroxy-acetaldehyde (p-PAC), L-phenylalanine (L-PHE), L-tyrosine (L-TYR), p-coumaric acid (p-CA). “*” indicates overexpressed enzymes; Aro10 and Pdc5 in boxes are knockouts, “fbr” indicates feedback-resistant.

### DETAILED DESCRIPTION OF THE DISCLOSURE

A class of tyramine containing hydroxycinnamic acid amides have now been shown to exhibit agonistic activity toward HNF4α (hepatocyte nuclear factor 4α), a global nuclear transcription factor that regulates expression of genes involved in maintaining balanced metabolism (homeostasis). By agonizing HNF4α activity, the plant specific tyramine derivatives find use in mitigating the adverse effects of free fatty acids, modulating metabolism, improving digestive health and addressing the underlying pathogenesis of metabolic disorders, such as nonalcoholic fatty liver disease, nonalcoholic steatohepatitis and type II diabetes mellitus. Accordingly, the present disclosure provides a recombinant host cell, extract, food product and method for the recombinant production of these bioactive plant metabolites.

As used herein, the bioactive plant metabolite of the disclosure is a tyramine containing hydroxycinnamic acid amide having the structure of Formula (I):

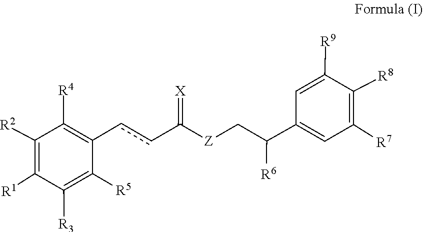

In some embodiments, R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, R¹, R², R³, and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, and R⁴, R⁵, R⁶, R⁷, and R⁹ are each independently hydrogen, deuterium, hydroxyl, or halogen;

In some embodiments, R¹, R², and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, and R³, R⁴, R⁵, R⁶, R⁷, and R⁹ are each independently hydrogen, deuterium, hydroxyl, or halogen.

In some embodiments, the dashed bond is present or absent.

In some embodiments, X is CH₂ or O.

In some embodiments, Z is CHR^{a}, NR^{a}, or O.

In some embodiments, Ra is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, a compound of Formula (I) is selected from (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, or 3-(3,4-dihydroxyphenyl)-N-(4-hydroxyphenethyl)propanamide, 3-(3,4-dihydroxyphenyl)-N-(4-(methylsulfonamido)phenethyl)propanamide.

In some embodiments, the bioactive plant metabolite of the disclosure is a tyramine containing hydroxycinnamic acid made having the structure of Formula (II):

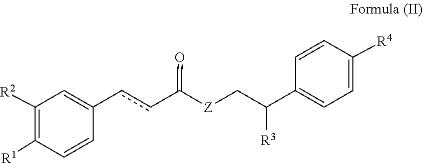

In some embodiments, R¹, R², R³, and R⁴ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, the dashed bond is present or absent.

In some embodiments, Z is CHR^{a}, NR^{a}, or O.

In some embodiments, Ra is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, a compound of Formula (II) is selected from (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, or 3-(3,4-dihydroxyphenyl)-N-(4-(methylsulfonamido)phenethyl)propanamide.

In some embodiments, the bioactive plant metabolite of the disclosure includes a tyramine containing hydroxycinnamic acid amide having the structure of Formula (III).

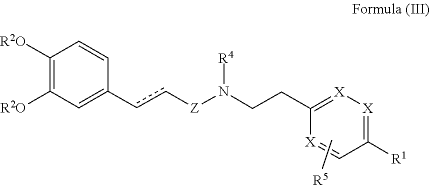

wherein

each occurrence of X may be independently C or N; Z may be —CR⁶— or —SO₂—R¹ may be selected from an OH, OCH₂CH₂R⁷, or NHR⁸ group, or R¹ together with R⁵ form a 6-membered substituted heterocycloalkyl ring, R² and R³ are independently selected from a hydrogen or CH₂CH₂R⁷ group, or R² and R³ together form a five- or six-membered heterocycloalkyl ring; R⁴ may be a hydrogen or CH₂CH₂R⁷ group; R⁵ may be present or absent and when present is a substituent on one or more ring atoms and for each occurrence is independently a halo, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl; R⁶ may be H₂, oxo, substituted alkyl, spirocycloalkyl or spiroheterocycloalkyl; R⁷ is a hydrogen, hydroxy, alkyl, substituted alkyl, alkoxy, substituted sulfonyl, carboxyl ester, amino, substituted amino, cyano, aryl, substituted aryl, cycloalkyl, heteroaryl, substituted heteroaryl; R⁸ may be substituted sulfonyl, substituted alkyl, carboxyl ester or aminocarbonyl; and the dashed bond may be present or absent.

In some embodiments, the bioactive plant metabolite of the disclosure includes a tyramine containing hydroxycinnamic acid amide having the structure of Formula (IV).

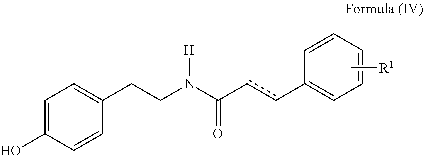

wherein

R¹ is present or absent, and when present is a substituent on one or more ring atoms (e.g., position 2, 3, and/or 4) and is for each ring atom independently a hydroxy group, halo group, substituted or unsubstituted lower alkyl group, or substituted or unsubstituted lower alkoxy group; and the dashed bond is present or absent. In accordance with this disclosure, a tyramine containing hydroxycinnamic acid amide includes both cis and trans isomers.

For the groups herein, the following parenthetical subscripts further define the groups as follows: “(Cₙ)” defines the exact number (n) of carbon atoms in the group. For example, “C₁-C₆-alkyl” designates those alkyl groups having from 1 to 6 carbon atoms (e.g., 1, 2, 3, 4, 5, or 6, or any range derivable therein (e.g., 3-6 carbon atoms)).

The term “lower alkyl” is intended to mean a branched or unbranched saturated monovalent hydrocarbon radical containing 1 to 6 carbon atoms (i.e., C₁-C₆-alkyl), such as methyl, ethyl, propyl, isopropyl, tert-butyl, butyl, n-hexyl and the like.

Similarly, a lower alkoxy group is a C₁-C₆-alkoxy group having the structure —OR wherein R is “alkyl” as defined further above. Particular alkoxy groups include, by way of example, methoxy, ethoxy, n-propoxy, isopropoxy, n-butoxy, tert-butoxy, iso-butoxy, sec-butoxy, n-pentoxy, 1,2-dimethylbutoxy, and the like.

The term “halo” is used herein to refer to chloro (Cl), fluoro (F), bromo (Br) and iodo (I) groups. In particular embodiments, the halo group is a fluoro group.

In any of the groups described herein, a substituted group (e.g., a substituted lower alkyl group or substituted lower alkoxy group) refers to an available hydrogen being replaced with an alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, alkoxyalkoxy, acyl, halo, nitro, cyano, carboxy, aralkoxycarbonyl, heteroarylsulfonyl, alkoxycarbonyl, alkylsulfonyl, alkylthio, arylthio, aryloxycarbonyl, arylsulfonyl, heteroarylthio, aralkylthio, heteroaralkylthio, cycloalkyl, heterocyclyl or glycosyl group.

Any undefined valency on an atom of a structure shown in this application implicitly represents a hydrogen atom bonded to the atom.

In some embodiments, the tyramine containing hydroxycinnamic acid amide has a structure of Formula (V):

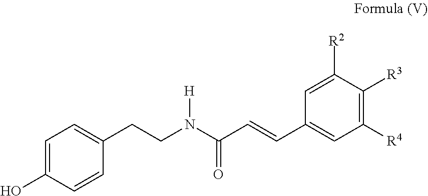

wherein,

R² is present or absent, and when present is a hydroxy or methoxy group;

R³ is present or absent, and when present is a hydroxy group; and

R⁴ is present or absent, and when present is a hydroxy or methoxy group.

“Isomer” refers to especially optical isomers (for example essentially pure enantiomers, essentially pure diastereomers, and mixtures thereof) as well as conformation isomers (i.e., isomers that differ only in their angles of at least one chemical bond), position isomers (particularly tautomers), and geometric isomers (e.g., cis-trans isomers).

In certain embodiments, the tyramine containing hydroxycinnamic acid amide of Formula (I)-(V) is selected from:

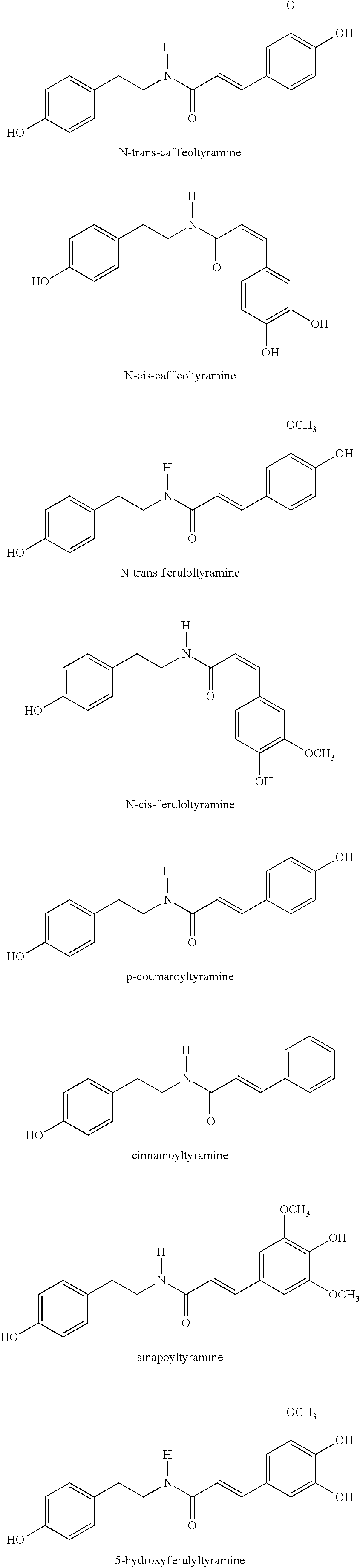

The tyramine containing hydroxycinnamic acid amides of this disclosure have been found in a number of plant genera including Solanum sp. (e.g., tomato, potato, nettle, chili pepper, and eggplant), Allium sp. (e.g., garlic, onion, and leek), Tribulus sp. (e.g., puncture vine) and Annona sp. (e.g., cherimoya, custard apple and sweetsop). In general, the biosynthetic approach of this disclosure may be carried out as depicted in Scheme 1.

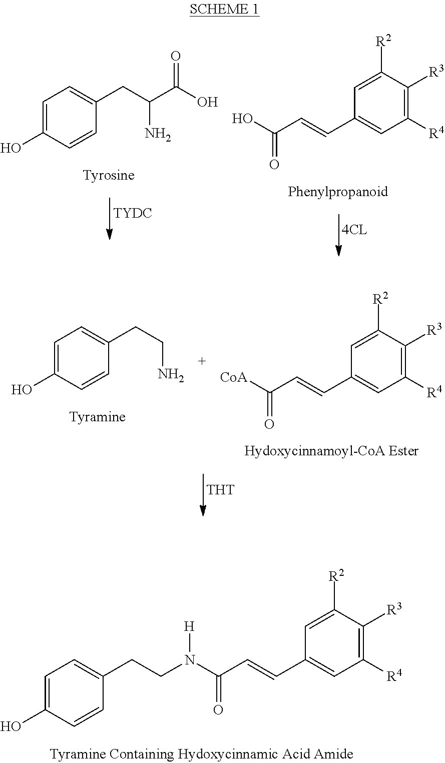

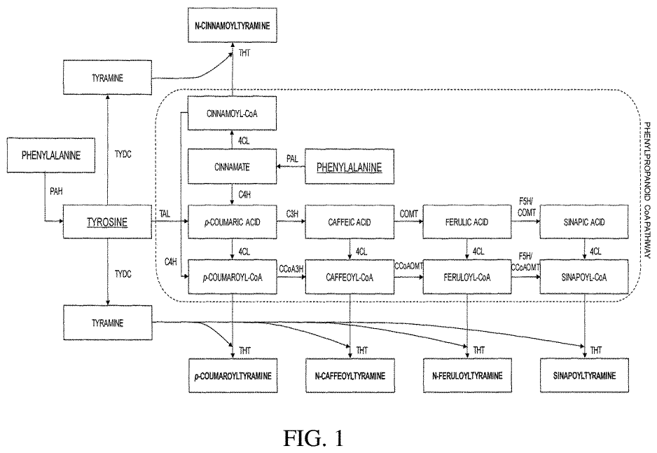

More specifically, the biosynthetic pathway of the tyramine containing hydroxycinnamic acid amides of this disclosure is presented in FIG. 1. As illustrated in FIG. 1, a recombinant host cell is provided which is capable of producing a tyramine containing hydroxycinnamic acid amide, wherein the host cell overproduces L-tyrosine and/or L-phenylalanine and includes one or more nucleic acid molecules encoding one or more enzymes of a phenylpropanoid CoA pathway for making a hydroxycinnamoyl-CoA ester; a nucleic acid molecule encoding a tyrosine decarboxylase (E.C. 4.1.1.25); and an exogenous nucleic acid molecule encoding a tyramine N-hydroxycinnamoyltransferase (E.C. 2.3.1.110).

### Tyrosine and Phenylalanine Overproduction

A host cell exhibiting “overproduction of L-tyrosine or L-phenylalanine” refers to a cell that has been genetically modified to produce increased amounts of L-tyrosine, L-phenylalanine or both L-tyrosine and L-phenylalanine as compared to a wild-type cell. As used herein, the terms “phenylalanine,” “L-phenylalanine,” “Phe” and “L-Phe” are used interchangeably. Likewise, the terms “tyrosine,” “L-tyrosine,” “Tyr” and “L-Tyr” are used interchangeably.

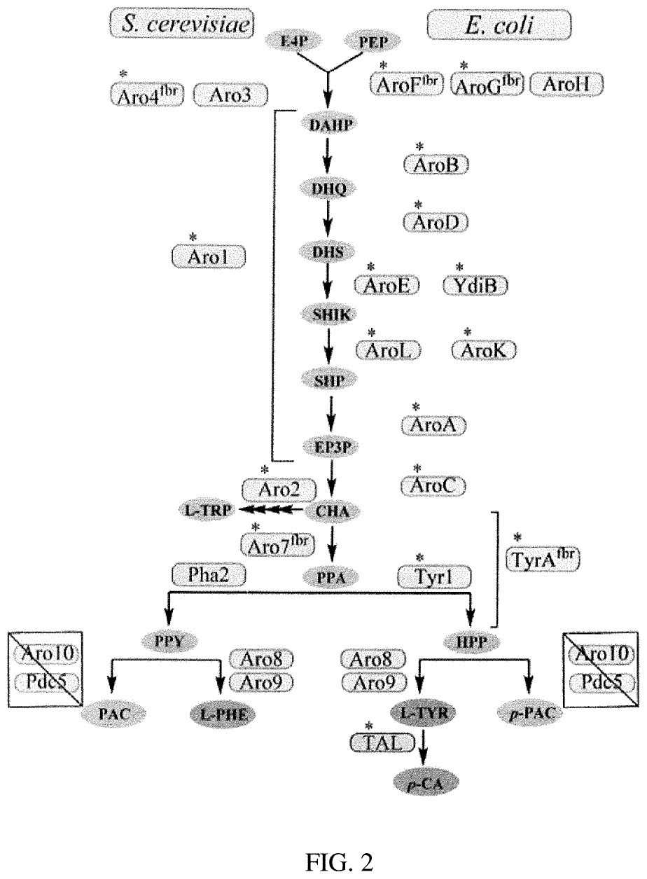

Many bacteria are natural producers of aromatic compounds via the shikimate pathway (Bongaerts, et al. (2001) Metab. Eng. 3:289-300; Ikeda, et al. (2006) Appl. Microbial. Biotechnol. 69:615-626; Sprenger, et al. (2007) Appl. Microbial. Biotechnol. 75:739-749). In this pathway, phosphoenolpyruvate (PEP) and erythrose 4-phosphate (E4P) converted from glucose through the central metabolic pathway are initially combined to form 3-deoxy-d-arabino-heptulosonate-7-phosphate (DAHP), which is then converted to chorismate. From chorismate, the pathway branches to form a variety of aromatic end products, including phenylalanine (Phe), tyrosine (Tyr), and tryptophan (Trp) (FIG. 2). To enhance the productivity of aromatic compounds, genetically modified strains have been generated. For example, in Phe production, the most important steps are the first and last steps of the shikimate pathway. However, enzymes including these steps, DAHP synthase (encoded by aroG) and chorismate mutase/prephenate dehydrates (encoded by pheA), are strongly inhibited by Phe. Therefore, feedback-resistant mutants (fbr) have been studied and exploited for Phe production (Kikuchi, et al. (1997) Appl. Environ. Microbiol. 63:761-762; Nelms, et al. (1992) Appl. Environ. Microbiol. 58:2592-2598). In addition, the levels of expression of both aroG and pheA are controlled by the transcriptional repressor TyrR so that deletion of tyrR is also efficient for Phe production (Berry (1996) Trends Biotechnol. 14:250-256). To enhance the availability of the DAHP precursors PEP and E4P, transketolase (tktA) and PEP synthase (pps) genes have been overexpressed (Patnaik & Liao (1994) Appl. Environ. Microbiol. 60:3903-3908), the PEP carboxylase gene (ppc) has been deleted (Miller, et al. (1987) J. Ind. Microbiol. 2:143-149), the carbon storage regulator genes (csrA or csrB) have been overexpressed or deleted (Tatarko & Romeo (2001) Curr. Microbiol. 43:26-32; Yakandawala, et al. (2008) Appl. Microbiol. Biotechnol. 78:283-291), and the glucose transport system has been exchanged from PEP-dependent sugar phosphotransferase system (PTS) to either galactose permease (GalP) -glucokinase (Glk) system (Baez-Viveros, et al. (2004) Biotechnol. Bioeng. 87:516-524; Yi, et al. (2003) Biotechnol. Prog. 19:1450-1459) or Zymomonas mobilis glucose facilitator (Glf) -Glk system (Patnaik & Liao (1994) Appl. Environ. Microbiol. 60:3903-3908). These modifications have been used in suitable combinations to enhance the production of aromatic compounds. To redirect synthesis to L-tyrosine, the pheA gene encoding chorismate mutase/prephenate dehydratase, has been deleted and tyrA, encoding chorismate mutase/prephenate dehydrogenase, has been inserted with a strong trc promoter to achieve an L-Tyr titer of 55 g/L in 48 hours (Olsen, et al. (2007) Appl. Microbiol. Biotechnol. 74(5):1031-40).

Exemplary bacterial strains for overproduction of tyrosine and/or phenylalanine include, but are not limited to, the strains listed in Table 1.

TABLE 1

| | | |
|---|---|---|
| Strain Name | | |
| (Main compound | | |
| overproduced) | Characteristics | Reference |

E. coli

| | | |
|---|---|---|
| 22A and 22A75D | Evolved from BY4741 | McKenna, et al. |
| (Phe) | | (2014) Microb. Cell Fact. |
| | | 13: 123 |
| AR-G91 | tyrR::T7p-aroF^{fbr}^{_{—}} | Koma et al. |
| (Phe) | pheA^{fbr} | (2012) Appl. Microbiol. |
| | | Biotechnol. |
| | | 93: 815-829 |
| W14/pR15BABKG | Δcrr ΔtyrA/(plasmid) | Liu, et al. |
| (Phe) | P_{R} aroG15 tyrB, P_{L} | (2014) Process Biochem. |
| | pheA^{fbr} ydiB aroK yddG | 49: 751-757 |
| FUS4.11/pF81ₖₐₙ | ΔpheA ΔtyrA ΔaroF | Weiner, et al. |
| (Phe) | ΔlacIZYA ΔpykA | (2014) Biochem. Eng. J. |
| | ΔpykF/(chromosome) P_{tac} | 83: 62-69 |
| | aroF aroB aroL, | |
| | (plasmid) P_{tac} pheA^{fbr} | |
| | aroF aroB aroL | |
| BL21 (DE3) | (plasmid) containing | Khamduang, et al. |
| (Phe) | the phenylalanine | (2009) J. Ind. Microbiol. |
| | dehydrogenase gene of | Biotechnol. |
| | Acinetobacter lwoffii | 36: 1267-1274 |
| AR-G2 | tyrR::T7p-aroF^{fbr}^{_{—}} | Koma et al. |
| (Tyr) | tyrA^{fbr} | (2012) Appl. Microbiol. |
| | | Biotechnol. |
| | | 93: 815-829 |
| MG1655 | (plasmid) P_{lac-UV5} aroE | Juminaga, et al. |
| derivative | aroD aroB^{op}, P_{L-tetO1} | (2012) Appl. Environ. |
| (Tyr) | aroG^{fbr} ppsA tktA, | Microbiol. |
| | (plasmid) P_{lac-UV5} tyrB | 78(1): 89-98 |
| | tyrA^{fbr} aroC, P_{trc} aroA | |
| | aroL | |
| rpoA 14^{R} | ΔpheA ΔtyrR/ | Santos, et al. |
| | (chromosome) P_{L} tyrA^{fbr} | (2012) Proc. Natl. Acad. |
| | aroG^{fbr}, point mutations in | Sci. USA |
| | hisH and purF, (plasmid) rpoA | 109: 13538-13543 |
| MG1655 | ΔpheA ΔpheL/ | Patnaik, et al. |
| derivative | (chromosome) P_{trc} tyrA | (2008) Biotechnol. |
| (Tyr) | | Bioeng. |
| | | 99(4): 741-52 |

Pseudomonas putida

| | |
|---|---|
| CM12-5 | Molina-Santiago, et al. |
| (Phe) | (2016) Microbiology |
| | 162: 1535-43 |

In S. cerevisiae, aromatic compounds are synthesized via the aromatic amino acid biosynthetic pathway (AAP) (Braus (1991) Microbiol Rev. 55:349-70). This highly regulated pathway is a central node of yeast metabolism and feeds several other pathways (e.g., quinone, folate and Ehrlich pathways; FIG. 2). Using flux balance analysis, it has been shown that availability of erythrose-4-phosphate (E4P) can be achieved by deleting ZWF1 and overexpressing TKL1-encoding transketolase to reverse flux from the glycolytic intermediates fructose-6-phosphate (F6P) and glyceraldehyde-3-phosphate (G3P) to E4P and xylulose-5-phosphate (X5P). This resulted in an up to 7-fold increase of the flux into the aromatic amino acid pathway (APP; Deaner & Alper (2017) Metab. Eng. 40:14-22). Another approach to improve the flux toward the AAP is the overexpression of transaldolase (Tall) and enolase 2 (Eno2) (Mao, et al. (2017) Biotechnol. Lett. 39(7):977-982). Tall favors the conversion of sedoheptulose-7-phosphate (S7P) and G3P to E4P and F6P, while Eno2 converts 2-phosphoglycerate to phosphoenolpyruvate (PEP).

The first enzymatic step of the shikimic acid pathway is catalyzed by DAHP synthase, which condenses E4P and PEP into 3-deoxy-D-arabinoheptulosonate 7-phosphate (DAHP; FIG. 2). AR03 and AR04 encode for two DAHP synthase isoforms in yeast. Combined deletion of AR03, and overexpression of Aro4^{K229L} and AR07^{FBR} (to avoid feedback inhibition) increases the flux through the aromatic amino acid pathway (Luttik, et al. (2008) Metab. Eng. 10:141-153). Moreover, tyrosine-insensitive mutant Aro4^{G226S} improves the production of tyrosine-derived naringenin (Koopman, et al. (2012) Microb. Cell Fact. 11:155) and has been used for the production of tyrosine-derived opioids (Galanie, et al. (2015) Science 349:1095-100). It has been shown that the last step of the shikimate pathway, i.e., conversion of EPSP into chorismite by chorismate synthase (Aro2), is a bottleneck in the AAP. Accordingly, overexpression of Aro2 has been shown to provide a 2-fold improvement in the levels of p-coumaric acid from tyrosine (Mao, et al. (2017) Biotechnol. Lett. 39(7):977-982).

Further downstream, from chorismate toward the tyrosine and phenylalanine branch, a common enzymatic step catalyzed by Aro7 converts chorismate into prephenate (FIG. 2). Aro7 is the third feedback regulatable enzyme of the aromatic amino acid biosynthetic pathway. Mutation of Aro7 (e.g., Aro7^{G141S} or Aro7^{T226I}) has been shown to relieve feedback regulation in S. cerevisiae and improve the titers of the intermediates of tyrosine and phenylalanine pathway, when compared to the equally engineered strain overexpressing the wild-type isoform of Aro7 (Luttik, et al. (2008) Metab. Eng. 10:141-153; Trenchard, et al. (2015) Metab. Eng. 31:74-83).

The next reaction step is the conversion of prephenate to phenylpyruvate (PPY), precursor of phenylalanine, or to hydroxyphenylpyruvate (4-HPP), precursor of tyrosine. Tyr1 catalyzes the reaction to 4-HPP, and it has been shown that overexpression of Tyr1 in combination with upper pathway modifications increases the production of tyrosine-derived p-coumaric acid (Mao, et al. (2017) Biotechnol. Lett. 39(7):977-982).

Aro10 catalyzes the entrance reaction into the catabolism of amino acids, the Ehrlich pathway. By deletion of ARO10, PDC5 and PDC6, the titer of the Ehrlich pathway intermediate phenylethanol decreases by 22-fold in a strain producing the flavonoid naringenin (Koopman, et al. (2012) Microb. Cell Fact. 11:155).

Exemplary eukaryotic host cells for overproduction of tyrosine and/or phenylalanine include, but are not limited to, the strains listed in Table 2.

TABLE 2

| Strain Name | Characteristics | Reference |
|---|---|---|
| ST4050 | Aro10Δpdc5Δ | Rodriguez, et al. |
| | | (2015) Metabol. Engineer. |
| | | 31: 181-8 |
| ST3213 | Aro10Δpdc5Δ | Rodriguez, et al. |
| | AR04^{K229L}AR07^{G1415} | (2015) Metabol. Engineer. |
| | | 31: 181-8 |
| IMK393 | MATalpha ura3 his3 leu2 | Koopman, et al. |
| | trp1 MAL2-8cSUC2 | (2012) Microb. Cell Fact. |
| | Δaro3 (46, 1065)::loxP | 11: 155 |
| | AR04^{G226S} pdc6Δ | |
| | (−6, −2)::loxP pdc5Δ | |
| | (−6, −2)::loxP aro10Δ | |
| | (−6, −2)::loxP | |

### Phenylpropanoid CoA Pathway

In accordance with the present disclosure, one or more nucleic acid molecules encoding one or more enzymes of a phenylpropanoid CoA pathway are engineered into the recombinant host cell to produce a hydroxycinnamoyl-CoA ester from phenylalanine and/or tyrosine. As used herein, the term “phenylpropanoid CoA pathway” refers enzymatic pathways internal to a cell needed for the production of a hydroxycinnamoyl-CoA ester (i.e., p-coumaroyl-CoA, cinnamoyl-CoA, caffeoyl-CoA, feruloyl-CoA and sinapoyl-CoA), preferably from phenylalanine and/or tyrosine. As illustrated in FIG. 1, enzymes of the phenylpropanoid CoA pathway include phenylalanine ammonia lyase, 4-coumarate-CoA ligase, cinnamate-4-hydroxylase, coumarate-3-hydroxylase, coumaroyl-CoA 3-hydroxylase, caffeoyl-CoA O-methyltransferase, ferulate-5-hydroxylase, caffeic acid/5-hydroxyferulic acid O-methyltransferase, tyrosine ammonia lyase. More specifically, phenylalanine is converted to cinnamate by expressing a phenylalanine ammonia lyase (PAL; EC 4.3.1.24). Cinnamate (also known as trans-cinnamic acid, cinnamic acid or trans-cinnamate) is then converted to p-coumaric acid (also known as para-hydroxycinnamic acid, p-hydroxycinnamic acid, 4-hydroxycinnamic acid or 4-hydroxycinnamate) by expressing a cinnamate 4-hydroxylase (C4H; E.C. 1.14.14.91), a P450 enzyme. Coumaroyl CoA ligase (4CL; E.C. 6.2.1.12) converts p-coumaric acid (and other substituted cinnamic acids) into the corresponding CoA thiol esters (i.e., p-coumaroyl CoA), which are used for the biosynthesis of flavonoids, isoflavonoids, lignin, suberins, and coumarins (Ehlting, et al. (1999) Plant J. 19(1):9-20).

Phenylalanine ammonia lyases are widely distributed in plants (Koukol, et al. (1961) J. Biol. Chem. 236:2692-2698), fungi (Bandoni, et al. (1968) Phytochemistry 7: 205-207), yeast (Ogata, et al. (1967) Agric. Biol. Chem. 31:200-206), and Streptomyces (Emes, et al. (1970) Can. J. Biochem. 48:613-622), but have not been found in E. coli or mammalian cells (Hanson & Havir, In: The Enzymes (3^{rd} ed.) Boyer Ed., Academic: New York, 1967; pp 75-167). PAL enzymes convert phenylalanine to cinnamic acid, which can be further converted to p-coumaric acid by a cinnamate-4-hydroxylase (C4H, E.C. 1.14.14.91). Moreover, as C4H is a cytochrome P450 enzyme, a cytochrome P450 reductase (CPR) may also be coexpressed. Accordingly, in some embodiments, a host cell of the disclosure expresses a PAL enzyme in combination with a C4H enzyme. In another embodiment, a host cell of the disclosure expresses a PAL enzyme in combination with a C4H and CPR enzyme.

Phenylalanine ammonia lyases will, to some extent, also accept tyrosine as a substrate, converting tyrosine directly to p-coumaric acid. For example, PAL enzymes isolated from parsley (Appert, et al. (1994) Eur. J. Biochem. 225:491) or corn (Havir et al. (1971) Plant Physiol. 48:130) demonstrate the ability to use tyrosine as a substrate. Similarly, the PAL enzyme isolated from Rhodosporidium (Hodgins (1971) J. Biol. Chem. 246:2977) also may use L-tyrosine as a substrate. Such enzymes are referred to herein as “PAL/TAL” enzymes (E.C. 4.3.1.25; Rosier, et al. (1997) Plant Physiol. 113:175-179). As such, PAL enzymes (especially those having a PAL/TAL activity ratio of at least 0.1) can also be expressed by a host cell of this disclosure. Where it is desired to create a recombinant organism expressing a wild-type gene encoding PAL/TAL activity, genes are isolated from maize, wheat, parsley, Rhizoctonia solani, Rhodosporidium, Sporobolomyces pararoseus, Rhodosporidium, and Phanerochaete chrysosporium (see Hanson & Havir (1981) Biochem. Plants 7:577-625). By way of illustration, using an aromatic amino acid-overproducing strain of P. putida S12, the pal gene encoding the bifunctional PAL/TAL enzyme from Rhodosporidium toruloides has been shown to increase production of cinnamate (Nijkamp, et al. (2005) Appl. Microbiol. Biotechnol. 69:170-77) and p-coumaric acid (Nijkamp, et al. (2007) Appl. Microbiol. Biotechnol. 74:617-624). Similarly, PALs from Arabidopsis thaliana (AtPa11 or AtPa12) have been used for the conversion of phenylalanine to cinnamate in S. cerevisiae (Koopman, et al. (2012) Microb. Cell Fact. 11:155).

Another biosynthetic pathway leading to the production of p-coumaric acid is based on the use of an enzyme having TAL activity (E.C. 4.3.1.23). Instead of the two enzyme reactions used to convert phenylalanine to p-coumaric acid, TAL converts L-tyrosine directly into p-coumaric acid. Accordingly, in come embodiments, a host cell of the disclosure expresses a TAL enzyme.

The classification of PAL and TAL enzymes s primarily determined by the enzyme's activity toward each substrate, where classification is assigned based on the preferred substrate. TAL enzymes are defined as those that preferentially use L-tyrosine as a substrate, whereas PAL enzymes are defined as those that preferentially use L-phenylalanine as a substrate. However, these enzymes normally accept both L-tyrosine and L-phenylalanine as substrates, albeit to varying degrees. As such, in some embodiments, PAL and TAL enzymes are generally referred to as “PAL/TAL enzymes.”

In some embodiments, specificity for one substrate over another can be achieved by, e.g., mutating a naturally-occurring PAL gene into one that encodes an enzyme that preferentially uses L-tyrosine as a substrate (see U.S. Pat. No. 6,368,837 or 6,521,748). A variety of approaches may be used for the mutagenesis of the PAL/TAL enzyme. Suitable approaches for mutagenesis include error-prone PCR (Leung, et al. (1989) Techniques 1:11-15; Zhou, et al. (1991) Nucleic Acids Res. 19:6052-6052; Spee, et al. (1993) Nucl. Acids Res. 21:777-778), in vitro mutagenesis, and in vivo mutagenesis. Protein engineering may be accomplished by the method commonly known as “gene shuffling” (U.S. Pat. Nos. 5,605,793; 5,811,238; 5,830,721; and 5,837,458), or by rationale design based on three-dimensional structure and classical protein chemistry.

The source of the PAL, TAL or PAL/TAL enzyme as well as the C4H enzyme in the present disclosure can be obtained or derived from any naturally-occurring source. Examples of suitable PAL, TAL, PAL/TAL and C4H enzymes of use in this disclosure are listed in Table 3.

TABLE 3

| Enzyme | Source Organism | Accession No. |
|---|---|---|
| | Rhodotorula mucilaginosa | CAA31486 |
| | Amanita muscaria | CAA09013 |
| | Ustilago maydis | AAL09388 |
| | Arabidopsis thaliana | NP_181241, |
| | | NP_187645, |
| | | NP_196043 |
| Phenylalanine | Glycine max | NP_001236956 |
| ammonium lyase | Medicago sativa | CAA41169 |
| and/or | Rehmannia glutinosa | AAK84225 |
| Tyrosine | Petroselinium crispum | CAA57056 |
| ammonium lyase | Prunus avium | AAC78457 |
| (PAL, TAL, | Lithospernum erythrorhizon | BAA24929 |
| PAL/TAL) | Citrus limon | ABB67733 |
| | Rhodotorula glutinis | AHB63479 |
| | (see also U.S. Pat. No. 6,521,748) | |
| | Phanerochaete chrysosporium | ACW34079 |
| | Flavobacterium johnsoniae | WP_012023194 |
| | Herpetosiphon aurantiacus | WP_012189949 |
| | | WP_012189431 |
| Cinnamate 4- | Cicer arietinum | 081928 |
| hydroxylase | | |
| (C4H) | | |
| | Populus tremuloides | 024312 |
| | Camellia sinensis | AAT68775 |
| | Vigna radiata | P37115 |
| | Helianthus tuberosus | Q04468 |
| | Camptotheca acuminata | AAT39513 |
| | Arabidopsis thaliana | P92994, |
| | | AAP68314 |
| | Ruta graveolens | AAN63028 |
| | Glycine max | Q42797 |
| | Citrus sinensis | AAF66065 |
| cytochrome P450 | Arabidopsis thaliana | CAA46814 |
| reductase (CPR) | | CAA46815 |
| | Helianthus tuberosus | CAA81209 |
| | | CAA81210 |

Accession Nos. obtained from GENBANK or UniProtKB/Swiss-Prot.

In another aspect, L-phenylalanine is converted to L-tyrosine using an enzyme having phenylalanine hydroxylase (PAH, E.C. 1.14.16.1) activity. The L-tyrosine produced using a phenylalanine hydroxylase is then subsequently converted to p-coumaric acid using an enzyme having TAL activity. Accordingly, in some embodiments, a host cell of the disclosure expresses a PAH enzyme in combination with a TAL enzyme. The PAH activity can be endogenous or introduced into the host cell to increase production of tyrosine. The PAH enzyme is well known in the art and has been reported in Proteobacteria (Zhao, et al. (1994) Proc. Natl. Acad. Sci. USA. 91:1366) For example, Pseudomonas aeruginosa possesses a multi-gene operon that includes phenylalanine hydroxylase (Zhao, et al. (1994) Proc. Natl. Acad. Sci. USA. 91:1366). The enzymatic conversion of L-phenylalanine to L-tyrosine is also known in eukaryotes. Human phenylalanine hydroxylase is specifically expressed in the liver to convert L-phenylalanine to L-tyrosine (Wang, et al. (1994) J. Biol. Chem. 269 (12): 9137-46). The source of the PAH enzyme in the present disclosure can be obtained or derived from any naturally-occurring source. Examples of suitable PAH enzymes of use in this disclosure are listed in Table 4.

TABLE 4

| Source Organism | Accession No. |
|---|---|
| Chromobacterium violaceum | AAA23115 |
| Pseudomonas aeruginosa | AAA25938 |
| Geodia cydonium | CAA76184 |
| Xanthomonas axonopodis | AAM35066 |
| Xanthomonas campestris | AAM39475 |
| Nocardia farcinica | BAD55786 |
| Gallus | NP_001001298 |

Accession Nos. obtained from GENBANK or UniProtKB/Swiss-Prot.

According to some embodiments, the host cell is engineered to recombinantly express nucleic acids encoding enzymes required to convert a portion of the aromatic amino acids overproduced by the host cell (L-phenylalanine and/or L-tyrosine) into p-coumaric acid by recombinantly expressing nucleic acids encoding (i) PAL and C4H, (ii) PAL, C4H and CPR, (iii) PAL/TAL and C4H, (iv) PAL/TAL, C4H and CPR, (v) TAL, and/or (vi) PAH and TAL of the phenylpropanoid pathway.

The p-coumaric acid produced by the recombinant host cell is converted into p-coumaroyl-CoA by expressing an enzyme having coumaroyl-CoA ligase activity. Coumaroyl-CoA ligases (4CL, E.C. 6.2.1.12) are used in the context of the present disclosure to catalyze the conversion of p-coumaric acid and other substituted cinnamic acids (e.g., cinnamate, caffeic acid, ferulic acid and sinapic acid) into the corresponding CoA thiol esters (i.e., p-coumaroyl-CoA, cinnamoyl-CoA, caffeoyl-CoA, feruloyl-CoA and sinapoyl-CoA). Coumaroyl-CoA ligases are well-known in the art. The coumaroyl-CoA ligase can be endogenous or exogenous to the host cell. In certain embodiments, the coumaroyl-CoA ligase is overexpressed within the host cell to increase p-coumaroyl-CoA production. A non-limited list of publicly available coumaroyl-CoA ligases of use in this disclosure is provided in Table 5.

TABLE 5

| Source Organism | Accession No. |
|---|---|
| Streptomyces coelicolor | CAB95894 |
| Allium cepa | AAS48417 |
| Populus tremuloides | AAC24503 |
| Amorpha fruticosa | AAL35216 |
| Populus tomentosa | AAL02145 |
| Nicotiana tabacum | BAA07828 |
| Pinus taeda | AAA92669 |
| Glycine max | AAL98709 |
| Arabidopsis thaliana | NP_188761 |
| | NP_176686 |
| | AAQ86590 |
| Rubus idaeus | AAF91310 |
| Lithospermum erythrorhizon | BAA08366 |
| Zea mays | AAS67644 |

Accession Nos. obtained from GENBANK or UniProtKB/Swiss-Prot.

In one aspect, the coumaroyl-CoA ligase is chosen based on its ability to convert p-coumaric acid into p-coumaroyl-CoA. In another aspect, a plurality of coumaroyl-CoA ligases are co-expressed to increase the production of tyramine containing hydroxycinnamic acid amides.

For production of caffeic acid or caffeoyl-CoA from p-coumaric acid or p-coumaroyl-CoA, respectively, the recombinant host cell may further include and express nucleic acids encoding a coumarate-3-hydroxylase (C3H, E.C. 1.14.13.-) or a coumaroyl-CoA 3-hydroxylase (CCoA3H, E.C. 1.14.14.96) Similarly, for production of ferulic acid or feruloyl-CoA from p-coumaric acid or p-coumaroyl-CoA, respectively, the recombinant host cell may further include and express nucleic acids encoding a coumarate-3-hydroxylase (C3H, E.C. 1.14.13.-) or a coumaroyl-CoA 3-hydroxylase (CCoA3H, E.C. 1.14.14.96), and a caffeic acid/5-hydroxyferulic acid O-methyltransferase (COMT, E.C. 2.1.1.68) or a caffeoyl-CoA O-methyltransferase (CCoAOMT, E.C. 2.1.1.104). To accelerate the conversion of caffeoyl-CoA to feruloyl-CoA, thereby increasing the rate of production of the final product, the host cell may be supplemented with S-adenosyl-methionine (AdoMet), be selected for overproduction of AdoMet (Choi, et al. (2009) Korean J. Chem. Eng. 26(1):156-9) or optionally be engineered to overproduce AdoMet. By way of illustration, a yeast strain expressing a chimeric protein composed of the yeast Met13p N-terminal catalytic domain and the Arabidopsis thaliana MTHFR (AtMTHFR-1)C-terminal regulatory domain was found to accumulate more than 100-fold more AdoMet than the wild type (Roje, et al. (2002) J. Biol. Chem. 277:4056-4061). Accordingly, in certain embodiments, the recombinant host cell overproduces AdoMet. Moreover, to synthesize sinapoyl-CoA, the recombinant host cell may express nucleic acids encoding a coumarate-3-hydroxylase (C3H, E.C. 1.14.13.-) or a coumaroyl-CoA 3-hydroxylase (CCoA3H, E.C. 1.14.14.96), a caffeic acid/5-hydroxyferulic acid O-methyltransferase (COMT, E.C. 2.1.1.68) or a caffeoyl-CoA O-methyltransferase (CCoAOMT, E.C. 2.1.1.104), and a ferulate-5-hydroxylase (F5H, E.C. 1.14.-.-). A non-limited list of publicly available enzymes for producing these hydroxycinnamoyl-CoA esters is provided in Table 6.

TABLE 6

| Enzyme | Source Organism | Accession No. |
|---|---|---|
| Coumarate-3-hydroxylase | Arabidopsis thaliana | NP_850337 |
| (C3H) | | |
| | Dendrobium officinale | KT239106 |
| | Oryza sativa | NP_001055922 |
| coumaroyl-CoA | Arabidopsis thaliana | NP_850337 |
| 3-hydroxylase (CCoA3H) | | |
| caffeic | Populus tremuloides | AAB61731 |
| acid/5-hydroxy ferulic | | |
| acid O-methyltransferase | | |
| (COMT) | | |
| | Glycine max | NP_001240003 |
| | Arabidopsis thaliana | NP_200227 |
| | Jatropha curcas | NP_001295701 |
| caffeoyl-CoA | Arabidopsis thaliana | NP_001328048 |
| O-methyltransferase | | AAM66108 |
| (CCoAOMT) | | |
| | Cicer arietinum | NP_001351681 |
| | Prunus mume | NP_001313438 |
| | Artemisia annua | PWA69367 |
| | Coffea canephora | ABO77959 |
| ferulate-5-hydroxylase | Arabidopsis thaliana | NP_195345 |
| (F5H) | | |
| | Trapa bicornis | ALC76578 |
| | Populus trichocarpa | CAB65335 |

Accession Nos. obtained from GENBANK or UniProtKB/Swiss- Prot.

### Production of Tyramine Containing Hydroxycinnamic Acid Amides

To convert the hydroxycinnamoyl-CoA esters (i.e., p-coumaroyl-CoA, cinnamoyl-CoA, caffeoyl-CoA, feruloyl-CoA and sinapoyl-CoA) to the corresponding tyramine containing hydroxycinnamic acid amides, the host cell also harbors and expresses a nucleic acid molecule encoding a tyramine N-hydroxycinnamoyltransferase (THT, E.C. 2.3.1.110). Tyramine N-hydroxycinnamoyltransferases are used in the context of the present disclosure to conjugate a hydroxycinnamoyl-CoA ester to tyramine to produce a tyramine containing hydroxycinnamic acid amide (i.e., N-caffeoyltyramine, N-feruloyltyramine, p-coumaroyltyramine, cinnamoyltyramine or sinapoyl tyramine). THTs are well-known in the art and can be endogenous or exogenous to the host cell. In certain embodiments, the THT is overexpressed within the host cell. A non-limited list of publicly available THT enzymes of use in this disclosure is provided in Table 7.

TABLE 7

| Source Organism | Accession No. |
|---|---|
| Nicotiana tabacum | P80969 |
| Capsicum baccatum | PHT30257 |
| Capsicum annuum | PHT64088 NP 001311493 |
| Solanum tuberosum | NP 001305481 |
| Solanum lycopersicum | NP 001234022 |
| Nicotiana attenuate | XP 019254384 |

Accession Nos. obtained from GENBANK or UniProtKB/Swiss- Prot.

In order to provide a source of tyramine, the present host cell further includes a nucleic acid molecule encoding a tyrosine decarboxylase (TYDC, E.C. 4.1.1.25). A tyrosine decarboxylase of use in the context of the present disclosure to converts tyrosine to tyramine. The TYDC can be endogenous or exogenous to the host cell and is preferably overexpressed within the host cell. A non-limited list of publicly available TYDC enzymes of use in this disclosure is provided in Table 8.

TABLE 8

| Source Organism | Accession No. |
|---|---|
| Enterococcus hirae | AAQ73505 |
| Lactobacillus brevis | AFP73381 |
| Actinidia chinesis | PSS05769 |
| Vitis vinifera | XP_003631850 |

Accession Nos. obtained from GENBANK or UniProtKB/Swiss- Prot.

As used herein, the term “recombinant host,” “recombinant host cell” or “host cell” is intended to refer to a host, the genome of which has been augmented by at least one incorporated DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host cell described herein is augmented through the stable introduction of one or more recombinant genes. However, autonomous or replicative plasmids or vectors can also be used within the scope of this disclosure. Moreover, the present disclosure can be practiced using a low copy number, e.g., a single copy, or high copy number (as exemplified herein) plasmid or vector.

Generally, the introduced DNA is not originally resident in the host that is the recipient of the DNA, but it is within the scope of the disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis.

The term “recombinant gene” or “recombinant nucleic acid molecule” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene may be a DNA sequence from another species, or may be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA.

A recombinant gene encoding a polypeptide described herein includes the coding sequence for that polypeptide, operably linked, in sense orientation, to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. The term “heterologous nucleic acid” as used herein, refers to a nucleic acid introduced into a recombinant host, wherein said nucleic acid is not naturally present in said host. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

“Regulatory region” or “regulatory sequence” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically includes at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., intrans, enhancers, upstream activation regions, transcription terminators, and inducible elements.

Promoters of use to drive expression of the relevant genes in a desired host cell are numerous and familiar to those skilled in the art. Expression in a host cell can be accomplished in a transient or stable fashion. Transient expression can be accomplished by inducing the activity of a regulatable promoter operably linked to the gene of interest. Stable expression can be achieved by the use of a constitutive promoter operably linked to the gene of interest. Virtually any promoter capable of driving these genes is suitable for the present disclosure including, but not limited to FBAIN, FBAINm, EXP, FBA1, GPAT, CYC1, HIS3, GAL1, GAL10, ADH1, PGK, PROS, GAPDH, ADCI, TRP1, URA3, LEU2, ENO, TPI; AOXI (particularly useful for expression in Pichia); and lac, trp, IPL, IPRR, T7, tac, and trc (particularly useful for expression in E. coli).

When the host cell is yeast, transcriptional and translational regions functional in yeast cells are provided, particularly from the host species (see, e.g., WO 2004/101757) The promoters can be obtained, for example, from genes in the glycolytic pathway, such as alcohol dehydrogenase, glyceraldehyde-3-phosphate-dehydrogenase, glyceraldehyde-3-phosphate O-acyltransferase, phosphoglycerate mutase, fructose-bisphosphate aldolase, phosphoglucose-isomerase, phosphoglycerate kinase, etc.; or regulatable genes such as acid phosphatase, lactase, metallothionein, glucoamylase, the translation elongation factor EFl-cx (TEF) protein (U.S. Pat. No. 6,265,185), ribosomal protein S7 (U.S. Pat. No. 6,265,185), etc. Any one of a number of regulatory sequences can be used, depending upon whether constitutive or induced transcription is desired, the efficiency of the promoter in expressing the open reading frame of interest, the ease of construction and the like.

Nucleotide sequences surrounding the translational initiation codon ‘ATG’ have been found to affect expression in yeast cells. If the desired polypeptide is poorly expressed in yeast, the genes can be modified nucleotide sequences of exogenous to include an efficient yeast

translation initiation sequence to obtain optimal gene expression. For expression in yeast, this can be done by site-directed mutagenesis of an inefficiently expressed gene by fusing it in-frame to an endogenous yeast gene, preferably a highly expressed gene.

Termination control regions may also be derived from various genes native to the preferred hosts. Optionally, a termination site may be unnecessary, however, it is most preferred if included. The termination region can be derived from the 3′ region of the gene from which the initiation region was obtained or from a different gene. A large number of termination regions are known and function satisfactorily in a variety of hosts (when utilized both in the same and different genera and species from where they were derived) The termination region usually is selected more as a matter of convenience rather than because of any particular property. Preferably, the termination region is derived from a yeast gene, particularly Saccharomyces, Schizosaccharomyces, Candida, Yarrowia or Kluyveromyces. The 3′-regions of mammalian genes encoding y-interferon and α-2 interferon are also known to function in yeast. Termination control regions may also be derived from various genes native to the preferred hosts. Optionally, a termination site may be unnecessary; however, it is most preferred if included. In one embodiment, the terminator is the terminator is selected from the group consisting of LIP2, PEX20, and XPR2.

One or more genes, for heterologous nucleic acids, can example one be combined or more in a recombinant nucleic acid construct in “modules” useful for tyramine containing hydroxycinnamic acid amide production. Combining a plurality of genes or heterologous nucleic acids in a module, facilitates the use of the module in a variety of species. For example, genes involved in the biosynthesis of L-tyrosine and/or L-phenylalanine, a hydroxycinnamoyl-CoA ester, tyramine and a tyramine containing hydroxycinnamic acid amide can be combined such that each coding sequence is operably linked to a separate regulatory region, to form a tyramine containing hydroxycinnamic acid amide module for production in eukaryotic organisms. Alternatively, the module can express a polycistronic message for production of a tyramine containing hydroxycinnamic acid amide in prokaryotic hosts such as species of Rodobacter, E. coli, Bacillus or Lactobacillus. In addition to genes useful for tyramine containing hydroxycinnamic acid amide production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., acids, there is more than one nucleotide for many triplet amino that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into constructing modules constructs.

Standard recombinant DNA and molecular cloning techniques can be used to prepare the construct(s) and recombinant host cell of this disclosure. See, e.g., Sambrook, et al. (1989) Molecular Cloning: A Laboratory Manual, 2nd ed., Cold Spring Harbor Laboratory, Cold Spring Harbor, N.Y.; Silhavy, et al. (1984) Experiments with Gene Fusions, Cold Spring Harbor Laboratory, Cold Spring Harbor, N.Y.; and Ausubel, et al., (1987) In Current Protocols in Molecular Biology, Wiley-Interscience.

The present disclosure provides a tyramine containing hydroxycinnamic acid amide-producing recombinant host cell harboring nucleic acids encoding enzymes for the overproduction of L-tyrosine and/or L-phenylalanine, biosynthesis of hydroxycinnamoyl-CoA ester and tyramine precursors, as well as a tyramine N-hydroxycinnamoyltransferase for producing the tyramine containing eukaryotic hydroxycinnamic host cells are acid amide. Prokaryotic and both contemplated for use according to the disclosure as are single cells and cells in a cell culture, e.g., cell lines. Examples of suitable cells include bacterial host cells such as Escherichia coli or Bacillus sp.; yeast host cells, such as Saccharomyces cerevisiae; insect host cells, such as Spodoptera frugiperda; or human host cells, such as HeLa and Jurkat cells. Preferred eukaryotic host cells are haploid cells, such as from Candida sp., Pichia sp. and Saccharomyces sp. While bacterial host cells can be used, it is preferred that the present disclosure employs the use of a eukaryotic host cell, in particular a yeast host cell from the genera Saccharomyces, Kluyveromyces, Pichia, Hansenular Schizosaccharomyces, kluyveromyces, Yarrowia and Candida. S. cerevisiae has several attractive characteristics as a metabolic engineering platform for production of the compounds of this disclosure. In addition to its excellent accessibility to molecular and synthetic biology techniques, its eukaryotic nature facilitates functional expression of plant-derived biosynthetic genes. For example, S. cerevisiae can functionally express cytochrome P450-containing enzymes and its subcellular compartmentation is comparable to that of plant cells. Finally, its GRAS (generally recognized as safe) status facilitates subsequent application for the production of compounds for use in mammals. Accordingly, in certain embodiments, the host cell is preferably a eukaryotic host cell, most preferably S. cerevisiae.

Microbial expression systems and expression vectors containing regulatory sequences that direct high level expression of foreign proteins are well-known to those skilled in the art. Any of these could be used to construct chimeric genes for production of a tyramine containing hydroxycinnamic acid amide in the host cell. These chimeric genes could then be introduced into appropriate microorganisms via transformation to allow for expression of high level of the enzymes.

Once an appropriate expression construct has been prepared for expression in a host cell, it is placed in a plasmid vector capable of autonomous replication in a host cell or it is directly integrated into the genome of the host cell. Integration of expression cassettes can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

Where two or more genes are expressed from separate replicating vectors, it is desirable that each vector has a different means of selection and should lack homology to the other constructs to maintain stable expression and prevent reassortment of elements among constructs. Judicious choice of regulatory regions, selection means and method of propagation of the introduced construct can be experimentally determined so that all introduced genes are expressed at the necessary levels to provide for synthesis of the desired products.

Constructs harboring a coding region of interest may be introduced into a host cell by any standard technique. These techniques include transformation (e.g., lithium acetate transformation [Guthrie, C., Methods in Enzymology, 194:186-187 (1991)]), protoplast fusion, biolistic impact, electroporation, microinjection, or any other method that introduces the gene of interest into the host cell.

For convenience, a host cell that has been manipulated by any method to take up a DNA sequence (e.g., an expression cassette) will be referred to as “transformed” or “recombinant” herein. The transformed host will have at least one copy of the expression construct and may have two or more, depending upon whether the gene is integrated into the genome, amplified, or is present on an extrachromosomal element having multiple copy numbers. The transformed host cell can be identified by selection for a marker contained on the introduced construct.

Alternatively, a separate marker construct may be co-transformed with the desired construct, as many transformation techniques introduce many DNA molecules into host cells. Typically, transformed hosts are selected for their ability to grow on selective media. Selective media may incorporate an antibiotic or lack a factor necessary for growth of the untransformed host, such as a nutrient or growth factor. An introduced marker gene may confer antibiotic resistance or encode an essential growth factor or enzyme, thereby permitting growth on selective media when expressed in the transformed host. Selection of a transformed host can also occur when the expressed marker protein can be detected, either directly or indirectly. The marker protein may be expressed alone or as a fusion to another protein. The marker protein can be detected by its enzymatic activity (e.g., β-galactosidase can convert the substrate X-gal [5-bromo-4-chloro-3-indolyl-β-D-galactopyranoside] to a colored product, and luciferase can convert luciferin to a light-emitting product); or its light-producing or modifying characteristics (e.g., the green fluorescent protein when illuminated with of Aequorea Victoria fluoresces blue light). Alternatively, antibodies can be used to detect the marker protein or a molecular tag on, for example, a protein of interest. Cells expressing the marker protein or tag can be selected, for example, visually, or by techniques such as FACS or panning using antibodies. For selection of yeast transformants, any marker that functions in yeast may be used. Preferred for use herein are resistance to kanamycin, hygromycin and the aminoglycoside G418, as well as ability to grow on media lacking uracil or leucine.

In addition to a recombinant host cell, this disclosure also includes a method for producing a tyramine containing hydroxycinnamic acid amide using the recombinant host cell. In accordance with the method of this disclosure, a recombinant eukaryotic host cell capable of producing a tyramine containing hydroxycinnamic acid amide is provided and cultivated for a time sufficient for said recombinant eukaryotic host cell to produce the tyramine containing hydroxycinnamic acid amide. Once produced, the tyramine containing hydroxycinnamic acid amide is isolated from the recombinant eukaryotic host cell or from the cultivation supernatant. In general, media conditions which may be optimized for high-level expression of a particular coding region of interest include the type and amount of carbon source, the type and amount of nitrogen source, the carbon-to-nitrogen ratio, the oxygen level, growth temperature, pH, length of the biomass production phase and the time of cell harvest. Microorganisms of interest, such as yeast, are grown in complex media (e.g., yeast extract-peptone-dextrose broth (YPD)) or a defined minimal media that lacks a component necessary for growth and thereby forces selection of the desired expression cassettes (e.g., Yeast Nitrogen Base (DIFCO Laboratories, Detroit, Mich.)).

Fermentation or cultivation media in the present disclosure must contain a suitable carbon source for the production of a tyramine containing hydroxycinnamic acid amide. Suitable carbon sources may include, but are not limited to: monosaccharides (e.g., glucose, fructose), disaccharides (e.g., lactose, sucrose), oligosaccharides, polysaccharides (e.g., starch, cellulose or mixtures thereof), sugar alcohols (e.g., glycerol) or mixtures from renewable feedstocks (e.g., cheese whey permeate, cornsteep liquor, sugar beet molasses, barley malt). Additionally, carbon sources may include alkanes, fatty acids, esters of fatty acids, monoglycerides, diglycerides, triglycerides, phospholipids and various commercial sources of fatty acids including vegetable oils (e.g., soybean oil) and animal fats. Additionally, the carbon source may include one-carbon sources (e.g., carbon dioxide, methanol, formaldehyde, formate, carbon-containing amines) for which metabolic conversion into key biochemical intermediates has been demonstrated. Hence, it is contemplated that the source of carbon utilized in the present disclosure may encompass a wide variety of carbon-containing sources and will only be limited by the choice of the host organism. Although all of the above-mentioned carbon sources and mixtures thereof are expected to be suitable in the present disclosure, preferred carbon sources are sugars and/or fatty acids. Most preferred is glucose and/or fatty acids containing between 10-22 carbons.

Nitrogen may be supplied from an inorganic (e.g., (NH₄)₂SO₄) or organic source (e.g., urea or glutamate). In addition to appropriate carbon and nitrogen sources, the fermentation media must also contain suitable minerals, salts, cofactors, buffers, vitamins, and other components known to those skilled in the art suitable for the growth of the microorganism.

Alternatively, or in addition to the production of a tyramine containing hydroxycinnamic acid amide from a carbon source (e.g., glucose or molasses), this disclosure also provides for exogenous supplementation of a fermenter medium with one or more substrates intermediate to the biosynthetic pathway for producing the tyramine containing hydroxycinnamic acid amide. Accordingly, in a further aspect, L-phenylalanine, L-tyrosine, cinnamate, p-coumaric acid, caffeic acid, ferulic acid, sinapic acid and/or S-adenyl-L-methionine can be exogenously supplied to a recombinant host cell of this disclosure. One of skill in the art will recognize that there is a need to balance the carbon flow from aromatic amino acid production into production of a tyramine containing hydroxycinnamic acid amide so that a decrease in concentration of the free aromatic amino acids is not detrimental to the viability or health of the recombinant host cell. Thus, in some embodiments, L-phenylalanine and/or L-tyrosine can be exogenously supplemented to the culture medium to increase production of a tyramine containing hydroxycinnamic acid amide.

Recombinant host cells of this disclosure may be cultured using methods known in the art. For example, the cells may be cultivated by shake flask cultivation, small-scale or large-scale fermentation in laboratory or industrial fermenters performed in a suitable medium and under conditions allowing expression of the coding region of interest. Where commercial production of a tyramine containing hydroxycinnamic acid amide is desired a variety of fermentation methodologies may be applied. For example, large-scale production of a specific gene product over-expressed from a recombinant host may be produced by a batch, fed-batch or continuous fermentation process.

A batch fermentation process is a closed system wherein the media composition is fixed at the beginning of the process and not subject to further additions beyond those required for maintenance of pH and oxygen level during the process. Thus, at the beginning of the culturing process the media is inoculated with the desired organism and growth or metabolic activity is permitted to occur without adding additional sources (i.e., carbon and nitrogen sources) to the medium. In batch processes the metabolite and biomass compositions of the system change constantly up to the time the culture is terminated. In a typical batch process, cells proceed through a static lag phase to a high growth log phase and finally to a stationary phase, wherein the growth rate is diminished or halted. Left untreated, cells in the stationary phase will eventually die. A variation of the standard batch process is the fed-batch process, wherein the source is continually added to the fermenter over the course of the fermentation process. A fed-batch process is also suitable in the present disclosure. Fed-batch processes are useful when catabolite repression is apt to inhibit the metabolism of the cells or where it is desirable to have limited amounts of source in the media at any one time. Measurement of the source concentration in fed-batch systems is difficult and therefore may be estimated on the basis of the changes of measurable factors such as pH, dissolved oxygen and the partial pressure of waste gases (e.g., CO2). Batch and fed-batch culturing methods are common and well known in the art and examples Biotechnology: A may be Textbook found in Thomas D. Brock of Industrial Microbiology, in 2^{nd} ed., (1989) Sinauer Deshpande, Mukund V., (1992). Associates Sunderland, Mass.; or Appl. Biochem. Biotechnol., 36:227

Commercial production of a tyramine containing hydroxycinnamic acid amide may also be accomplished by a continuous fermentation process, wherein a defined media is continuously added to a bioreactor while an equal amount of culture volume is removed simultaneously for product recovery. Continuous cultures generally maintain the cells in the log phase of growth Continuous or semi-continuous modulation of one factor or affect cell growth or end at a constant cell density. culture methods permit the any number of factors that product concentration. For example, one approach may limit the carbon source and allow all other parameters to moderate metabolism. In other systems, a number of factors affecting growth may be altered continuously while the cell concentration, measured by media turbidity, is kept constant. Continuous systems strive to maintain steady state growth and thus the cell growth rate must be balanced against cell loss due to media being drawn off the culture. Methods of modulating nutrients and growth factors for continuous culture processes, as well as techniques for maximizing the rate of product formation, are well known in the art of industrial microbiology.

A tyramine containing hydroxycinnamic acid amide can be extracted from the host cell or from the cultivation supernatant by solvent extraction (e.g., partitioning) or precipitation, treatment with activated charcoal, evaporation, filtration, chromatographic fractionation, or a combination thereof. Solvent extraction may be carried out using, e.g., n-pentane, hexane, butane, chloroform, dichloromethane, di-ethyl ether, acetonitrile, water, butanol, isopropanol, ethanol, methanol, glacial acetic acid, acetone, norflurane (HFA134a), ethyl acetate, dimethyl sulfoxide, heptafluoropropane (HFA227), and subcritical or supercritical fluids such as liquid carbon dioxide and water, or a combination thereof in any proportion. When solvents such as those listed above are used, the resultant extract typically contains non-specific lipid-soluble material. This can be removed by a variety of processes including “winterization”, which involves chilling to a specified temperature, typically −20° C. followed by filtration or centrifugation to remove waxy ballast, extraction with subcritical or supercritical carbon dioxide or non-polar solvents (e.g., hexane) and by distillation.

Extracts enriched for a tyramine containing hydroxycinnamic acid amide are ideally obtained by chromatographic fractionation. Chromatographic fractionation typically includes column chromatography and may be based on molecular sizing, charge, solubility and/or polarity. Depending on the type of chromatographic method, column chromatography can be carried out with matrix materials composed of, for example, dextran, agarose, polyacrylamide or silica and can include solvents such as dimethyl sulfoxide, pyridine, water, dimethylformamide, methanol, saline, ethylene dichloride, chloroform, propanol, ethanol, isobutanol, formamide, methylene dichloride, butanol, acetonitrile, isopropanol, tetrahydrofuran, dioxane, chloroform/dichloromethane, etc.

Typically, the product of the chromatographic step is collected in multiple fractions, which may then be tested for the presence of the desired compound using any suitable analytical technique (e.g., thin layer chromatography, mass spectrometry) Fractions enriched in the desired compound may then be selected for further purification.

As an alternative, or in conjunction with chromatography, crystallization may be performed to obtain high purity tyramine containing hydroxycinnamic acid amides. The solubility of the tyramine containing hydroxycinnamic acid amide is adjusted by changing temperature and/or the composition of the solution, for instance by removing ethanol, and/or adjusting the pH to facilitate precipitation, followed by filtration or centrifugation of the precipitated crystals or oils.

By way of illustration, an extract comprising N-trans-caffeoyltyramine is obtained by subjecting the host cell or cultivation supernatant to 80% ethanol at room temperature, filtering and concentrating the 80% ethanol extract, resuspending the concentrated extract in water, partitioning the aqueous solution with hexane, adding chloroform to the aqueous layer, and subjecting the chloroform layer to liquid chromatography with silica gel. See, e.g., Ko, et al. (2015) Internatl. J. Mol. Med. 36(4):1042-8.

An extract comprising hydroxycinnamic acid amide can conventional techniques such as chromatography (HPLC) or high a tyramine containing be standardized using high-performance liquid performance thin-layer chromatography (HPTLC). The term “standardized extract” refers to an extract which is standardized by identifying characteristic ingredient(s) or bioactive marker(s) present in the extract. Characterization can be, for example, by analysis of the spectral data such as mass spectrum (MS), infrared (IR) and nuclear magnetic resonance (NMR) spectroscopic data.

A substantially pure tyramine containing hydroxycinnamic acid amide or extract comprising a tyramine containing hydroxycinnamic acid amide can be combined with a carrier and provided in any suitable form for consumption by or administration to a subject. Suitable consumable forms include, but are not limited to, a dietary supplement, food ingredient or additive, food product (e.g., a functional food), a medical food, nutraceutical or pharmaceutical composition.

A food ingredient or additive is an edible substance intended to result, directly or indirectly, in its becoming a component or otherwise affecting the characteristic of any food (including any substance intended for use in producing, manufacturing, packing, processing, preparing, treating, packaging, transporting, or holding food). A food product in particular a functional food, is a food fortified or enriched during processing to include additional complementary nutrients and/or beneficial ingredients. A food product according to this disclosure can, e.g. r be in the form of butter, margarine, sweet or savory spreads, biscuits, health bar, bread, cake, cereal, candy, confectionery, yogurt or a fermented milk product, juice-based and vegetable-based beverages, shakes, flavored waters, fermented beverage (e.g. Kombucha or fermented yerba mate), convenience snack such as baked or fried vegetable chips or other extruded snack products, or any other suitable food.

A dietary supplement is a product taken by mouth that contains a compound or extract of the disclosure and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease, or to affect the structure or any function of the body of humans or other animals. Dietary supplements, nutraceuticals and pharmaceutical compositions can be found in many forms such as tablets, coated tablets, pills, capsules, pellets, granules, softgels, gelcaps, liquids, powders, emulsions, suspensions, elixirs, syrup, and any other form suitable for use.

The phrase “carrier” as used herein means a material, composition or vehicle, such as a liquid or solid filler, diluent, excipient, manufacturing aid (e.g., lubricant, talc magnesium, calcium or zinc stearate, or steric acid), or sol vent encapsulating material, involved in carrying or transporting the subject compound from one organ, or portion of the body, to another organ, or portion of the body. Each carrier should be compatible with the other ingredients of the formulation and not injurious to the subject. Some examples of materials that can serve as carriers include: (1) sugars, such as lactose, glucose and sucrose; (2) starches, such as corn starch and potato starch; (3) cellulose, and its derivatives, such as sodium carboxymethyl cellulose, ethyl cellulose, cellulose acetate, and hydroxyl propyl methyl cellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (21) phospholipids and phospholipid derivatives; and (23) other non-toxic compatible substances employed in conventional formulations.

For preparing solid compositions such as tablets or capsules, the compound or extract is mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid composition. This solid composition is then subdivided into unit dosage forms containing an effective amount of the compound of the present disclosure. The tablets or pills containing the compound or extract can be coated or otherwise compounded to provide a dosage form affording the advantage of prolonged action and/or potentially enhanced absorption.

The liquid forms in which the compound or extract of the disclosure is incorporated for oral or parenteral administration include aqueous solution, suitably flavored syrups, aqueous or oil suspensions, and flavored emulsions with edible oils as well as elixirs and similar vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration may take the form of, for example, solutions, syrups or suspensions, or they may be presented as a dry product for reconstitution with water or other suitable vehicles before use. Such liquid preparations may be prepared by conventional means with acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

Methods of preparing formulations or compositions of this disclosure include the step of bringing into association a compound or extract of the present disclosure with the carrier and, optionally, one or more accessory and/or active ingredients. In general, the formulations are prepared by uniformly and intimately bringing into association a compound or extract of the present disclosure with liquid carriers, or finely divided solid carriers, or both, and then, if necessary, shaping the product. As such, the disclosed formulation may consist of, or consist essentially of a compound or extract described herein in combination with a suitable carrier.

When a compound or extract of the present disclosure is administered as pharmaceuticals, nutraceuticals, or dietary supplements to humans and animals, they can be given per se 0.1 to 99% or as a composition containing, for example, (more preferably, 10 to 30%) of active ingredient in combination with an acceptable carrier.

While it is contemplated that individual tyramine containing hydroxycinnamic acid amides may be used in the consumables of this disclosure, it is further contemplated that two or more of the compounds or extracts could be combined in any relative amounts to produce custom combinations of ingredients containing two or more tyramine containing hydroxycinnamic acid amides in desired ratios to enhance product efficacy, improve organoleptic properties or some other measure of quality important to the ultimate use of the product.

### Example 1: Recombinant Yeast Strains for Producing Tyramine Containing Hydroxycinnamic Acid Amides

Since tyramine containing hydroxycinnamic acid amides are not endogenous metabolites, it is necessary to recreate synthetic production pathways in yeast. Synthesis starts as all phenylpropanoids with phenylalanine and/or tyrosine, which are produced endogenously by the cell. Genes for introduction and overexpression in a recombinant yeast strain of the disclosure are listed in Table 9.

TABLE 9

| | | |
|---|---|---|
| | | Source of |
| Gene* | Description | Coding Sequence |

Amino Acid Biosynthesis

| | | |
|---|---|---|
| P_{PGK1}-scARO4^{fbr}^{_{—}}T_{CYC1} | Feedback resistant DAHP | S. cerevisiae |
| | synthase (K229L | (wild-type |
| | mutation) | Accession No. |
| | | DAA07365) |
| P_{TEF1}-scARO7^{fbr}^{_{—}}T_{ADH1} | Feedback resistant | S. cerevisiae |
| | chorismite mutase (G141S | (wild-type |
| | mutation) | Accession No. |
| | | NP_015385) |
| P_{TEF1}-scARO1-T_{ADH1} | Pentafunctional enzyme | S. cerevisiae |
| | converting DAHP to | (Accession No. |
| | 5-enolpyruvylshikimate-3- | NP_010412) |
| | phosphate (EPSP) | |
| P_{TEF1}-scTYR1-T_{ADH1} | Prephenate | S. cerevisiae |
| | Dehydrogenase | (Accession No. |
| | | NP_009725) |

Phenylpropanoid CoA Biosynthesis

| | | |
|---|---|---|
| P_{TPI1}-rtPAL-T_{CPS1} | Phenylalanine ammonia | Rhodosporidium |
| | lyase | toruloides |
| | | (Accession No. |
| | | CAA35886) |
| P_{PYK1}-atC4H-T_{DITl} | Cinnamate-4-hydroxylase | Arabidopsis |
| | | thaliana |
| | | (Accession No. |
| | | P92994) |
| P_{TPI1}-atCPR-T_{CPS1} | Cytochrome P450 | A. thaliana |
| | reductase | (Accession No. |
| | | CAA46814) |
| P_{TEF1}-atC4L-T_{CYC1} | 4-Coumarate-CoA ligase | A. thaliana |
| | | (Accession No. |
| | | NP_188761) |
| P_{TEF1}-atC3H-T_{RPL3} | Coumarate-3-hydroxylase | A. thaliana |
| | | (Accession No. |
| | | NP_850337) |
| P_{TEF1}-atCCoA3H-T_{RPL3} | coumaroyl-CoA | A. thaliana |
| | 3-hydroxylase | (Accession No. |
| | | NP_850337) |
| P_{TEF1}-atCCoAMT-T_{CYC1} | Caffeoyl-CoA | A. thaliana |
| | O-methyltransferase | (Accession No. |
| | | NP_001328048) |
| P_{TPI1}-atF5H-T_{ADH1} | Ferulate-5-hydroxylase | A. thaliana |
| | | (Accession No. |
| | | NP_195345) |
| P_{PYK1}-atCOMT-T_{CYC1} | Caffeic | A. thaliana |
| | acid/5-hydroxyferulic acid | (Accession No. |
| | O-methyltransferase | NP_200227) |

Tyramine Biosynthesis

| | | |
|---|---|---|
| P_{GPD1}-psTYDC-T_{CYC1} | Tyrosine decarboxylase | Papaver |
| | | Somniferum |
| | | (Accession No. |
| | | AAA97535) |

Tyramine Containing Hydroxycinnamic Acid Amide Biosynthesis

| | | |
|---|---|---|
| P_{TEF1}-caTHT-T_{CPS1} | Tyramine | Capsicum annuum |
| | N-hydroxycinnamoyl | (Accession No. |
| | transferase | NP_001311493) |

*Gene includes the promoter (“P”) sequence, coding sequence, and terminator (“T”) sequence.

Saccharomyces cerevisiae strains used are isogenic haploids. The starting yeast strain contains knock outs of auxotrophic (-ura3, -leu2, his3) marker genes. Enrichment and propagation of clones are made in YPD liquid cultures (10 g/l BACTO-yeast extract, 20 g/l BACTO-peptone and 2% dextrose) at 30° C. Recombinants are selected on dropout agar plates (YNB+CSM) in the absence of uracil or leucine or histidine. The gene defects in uracil, histidine and leucine biosynthetic pathway result in auxotrophy. For homologous recombination, a mismatch deficient strain is used. Open reading frames are synthesized and/or amplified by PCR.

Using convention cloning and yeast transformation protocols, constructs are introduced into yeast and cells are grown in medium with glucose as the sole carbon source. When additional substrates are required (e.g., phenylalanine, tyrosine or cinnamic acids), said substrates are added 24 hours after cultures are started. Supernatants are then analyzed by High performance liquid chromatography (HPLC) to identify the appropriate product.

In certain embodiments, the yeast cell overproduces one or both of phenylalanine and tyrosine. In particular embodiments, phenylalanine and tyrosine are produced by the recombinant host cells at approximately equal rates. In order to avoid production of aromatic alcohols and direct the pathway flux to aromatic amino acids, a double knockout of ARO10 (phenylpyruvate decarboxylase) and PDC5 (pyruvate decarboxylase) is introduced into the strain. Yeast strains for producing tyramine containing hydroxycinnamic acid amides as well as growth medium supplements are provided in Table 10.

TABLE 10

| Strain | | Growth | Main Product |
|---|---|---|---|
| Name | Characteristics | Medium | Produced |
| Y0001 | aro10Δ pdc5Δ ARO4^{FBR} | Glucose | p-coumaroyl-tyramine |
| | ARO7^{FBR} ARO1 TYDC | | |
| | PAL C4H CPR 4CL THT | | |
| Y0002 | aro10Δ pdc5Δ ARO4^{FBR} | Glucose | N-caffeoyl tyramine |
| | ARO7^{FBR} ARO1 TYDC | | |
| | PAL C4H CPR 4CL C3H | | |
| | THT | | |
| Y0003 | aro10Δ pdc5Δ ARO4^{FBR} | Glucose | N-feruloyl tyramine |
| | ARO7^{FBR} ARO1 TYDC | | |
| | PAL C4H CPR 4CL C3H | | |
| | CCoAOMT THT | | |
| Y0004 | aro10Δ pdc5Δ ARO4^{FBR} | Glucose | Sinapoyl tyramine |
| | ARO7^{FBR} ARO1 TYDC | | |
| | PAL C4H CPR 4CL C3H | | |
| | CCoA3H CCoAOMT F5H | | |
| | COMT THT | | |
| Y0005 | aro10Δ pdc5Δ ARO4^{FBR} | Glucose | N-cinnamoyl |
| | ARO7^{FBR} ARO1 TYDC | | tyramine |
| | PAL 4CL THT | | |
| Y0006 | aro10Δ pdc5Δ ARO4^{FBR} | Glucose, | p-coumaroyl-tyramine |
| | ARO7^{FBR} ARO1 TYDC | p-Coumaric | |
| | PAL C4H CPR 4CL THT | acid | |
| Y0007 | aro10Δ pdc5Δ ARO4^{FBR} | Glucose, | N-caffeoyl tyramine |
| | ARO7^{FBR} ARO1 TYR1 | Caffeic acid | |
| | TYDC 4CL THT | | |
| Y0008 | aro10Δ pdc5Δ ARO4^{FBR} | Glucose, | N-feruloyl tyramine |
| | ARO7^{FBR} ARO1 TYR1 | Ferulic acid | |
| | TYDC 4CL THT | | |
| Y0009 | aro10Δ pdc5Δ ARO4^{FBR} | Glucose, | Sinapoyl tyramine |
| | ARO7^{FBR} ARO1 TYR1 | Sinapic acid | |
| | TYDC 4CL THT | | |
| Y0010 | aro10Δ pdc5Δ ARO4^{FBR} | Glucose, | N-cinnamoyl |
| | ARO7^{FBR} ARO1 TYR1 | Cinnamate | tyramine |
| | TYDC 4CL THT | | |
| Y0011 | TYDC PAL C4H CPR | Glucose, | p-coumaroyl-tyramine |
| | 4CL THT | Phenylalanine, | |
| | | Tyrosine | |
| Y0012 | TYDC PAL C4H CPR | Glucose, | N-caffeoyl tyramine |
| | 4CL C3H THT | Phenylalanine, | |
| | | Tyrosine | |
| Y0013 | TYDC PAL C4H CPR | Glucose, | N-feruloyl tyramine |
| | 4CL C3H CCoAMT THT | Phenylalanine, | |
| | | Tyrosine | |
| Y0014 | TYDC PAL C4H CPR | Glucose, | Sinapoyl tyramine |
| | 4CL C3H CCoA3H | Phenylalanine, | |
| | CCoAOMT F5H COMT | Tyrosine | |
| | THT | | |
| Y0015 | TYDC PAL 4CL THT | Glucose, | N-cinnamoyl |
| | | Phenylalanine, | tyramine |
| | | Tyrosine | |

Strains exhibiting a high production level a tyramine containing hydroxycinnamic acid amide are used to produce extracts and consumables containing the tyramine containing hydroxycinnamic acid amide. Production strains are grown in bioreactors for a time sufficient to produce the tyramine containing hydroxycinnamic acid amide. Upon completion of the fermentation, the cell mass is removed from the supernatant by centrifugation or filtration. The tyramine containing hydroxycinnamic acid amide is then be recovered from the supernatant by extraction with a suitable solvent, for example, aqueous alcohol or ethyl acetate. The tyramine containing hydroxycinnamic acid amide may then be further purified by solvent partitioning and/or chromatography and crystallized by modifying the solvent for instance by adjusting the solution temperature and/or composition. The tyramine containing hydroxycinnamic acid amide may also be recovered directly from the cell mass by addition of ethanol or other suitable solvent, for instance ethyl acetate, by adding solvent directly to the cell culture, followed by filtration or centrifugation. After solvent removal from the supernatant, crystals (or other desolventized form such as an oil or precipitate) are collected. This material is then further purified by, for instance solvent partitioning and and/or chromatography, and crystalized by modifying the solvent's temperature and/or composition, yielding a high purity material which is then recovered, washed and dried to generate a purified (>90%) source of the tyramine containing hydroxycinnamic acid amide.

What is claimed is:

1. A recombinant eukaryotic host cell capable of producing a tyramine containing hydroxycinnamic acid amide, the recombinant host comprising:
(a) one or more nucleic acid molecules encoding one or more enzymes capable of overproduction of L-tyrosine or L-phenylalanine;
(b) one or more nucleic acid molecules encoding one or more enzymes of a phenylpropanoid CoA pathway for making a hydroxycinnamoyl-CoA ester;
(c) a nucleic acid molecule encoding a tyrosine decarboxylase (E.C. 4.1.1.25); and
(d) a nucleic acid molecule encoding a tyramine N-hydroxycinnamoyltransferase (E.C. 2.3.1.110).

2. The recombinant eukaryotic host cell of claim 1, wherein the one or more nucleic acid molecules encoding one or more enzymes of a phenylpropanoid CoA pathway for making a hydroxycinnamoyl-CoA ester comprising phenylalanine ammonia lyase, 4-coumarate-CoA ligase, cinnamate-4-hydroxylase, coumarate-3-hydroxylase, coumaroyl-CoA 3-hydroxylase, caffeoyl-CoA O-methyltransferase, ferulate-5-hydroxylase, caffeic acid/5-hydroxyferulic acid O-methyltransferase, tyrosine ammonia lyase, or a combination thereof.

3. The recombinant eukaryotic host cell of claim 1, wherein said host cell further overproduces methionine.

4. The recombinant eukaryotic host cell of claim 1, wherein the recombinant eukaryotic host cell is a recombinant yeast strain.

5. The recombinant eukaryotic host cell of claim 4, wherein the yeast strain is selected from:

| Strain Name | Characteristics |
|---|---|
| Y0001 | aro10Δ pdc5Δ ARO4^{FBR} |
| | ARO7^{FBR} ARO1 TYDC |
| | PAL C4H CPR 4CL THT |
| Y0002 | aro10Δ pdc5Δ ARO4^{FBR} |
| | ARO7^{FBR} ARO1 TYDC |
| | PAL C4H CPR 4CL C3H |
| | THT |
| Y0003 | aro10Δ pdc5Δ ARO4^{FBR} |
| | ARO7^{FBR} ARO1 TYDC |
| | PAL C4H CPR 4CL C3H |
| | CCoAOMT THT |
| Y0004 | aro10Δ pdc5Δ ARO4^{FBR} |
| | ARO7^{FBR} ARO1 TYDC |
| | PAL C4H CPR 4CL C3H |
| | CCoA3H CCoAOMT F5H |
| | COMT THT |
| Y0005 | aro10Δ pdc5Δ ARO4^{FBR} |
| | ARO7^{FBR} ARO1 TYDC |
| | PAL 4CL THT |
| Y0006 | aro10Δ pdc5Δ ARO4^{FBR} |
| | ARO7^{FBR} ARO1 TYDC |
| | PAL C4H CPR 4CL THT |
| Y0007 | aro10Δ pdc5Δ ARO4^{FBR} |
| | ARO7^{FBR} ARO1 TYR1 |
| | TYDC 4CL THT |
| Y0008 | aro10Δ pdc5Δ ARO4^{FBR} |
| | ARO7^{FBR} ARO1 TYR1 |
| | TYDC 4CL THT |
| Y0009 | aro10Δ pdc5Δ ARO4^{FBR} |
| | ARO7^{FBR} ARO1 TYR1 |
| | TYDC 4CL THT |
| Y0010 | aro10Δ pdc5Δ ARO4^{FBR} |
| | ARO7^{FBR} ARO1 TYR1 |
| | TYDC 4CL THT |
| Y0011 | TYDC PAL C4H CPR |
| | 4CL THT |
| Y0012 | TYDC PAL C4H CPR |
| | 4CL C3H THT |
| Y0013 | TYDC PAL C4H CPR |
| | 4CL C3H CCoAMT THT |
| Y0014 | TYDC PAL C4H CPR |
| | 4CL C3H CCoA3H |
| | CCoAOMT F5H COMT |
| | THT |
| Y0015 | TYDC PAL 4CL THT |

6. The recombinant eukaryotic host cell of claim 1, wherein the tyramine containing hydroxycinnamic acid amide is selected from at least one of N-caffeoyltyramine, N-feruloyltyramine, 5-hydroxyferuloyltyramine, cinnamoyltyramine, sinapoyltyramine, p-coumaroyltyramine, (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, or 3-(3,4-dihydroxyphenyl)-N-(4-(methylsulfonamido)phenethyl)propanamide.

7. The recombinant eukaryotic host cell of claim 6, wherein the tyramine containing hydroxycinnamic acid amide is N-caffeoyltyramine, N-feruloyltyramine, 5-hydroxyferuloyltyramine, cinnamoyltyramine, sinapoyltyramine, p-coumaroyltyramine.

8. A method for producing a tyramine containing hydroxycinnamic acid amide or a tyramine containing hydroxycinnamic acid derivative comprising:
(a) providing a recombinant eukaryotic host cell capable of producing a tyramine containing hydroxycinnamic acid amide, wherein said recombinant eukaryotic host cell overproduces L-tyrosine or L-phenylalanine and the recombinant eukaryotic host cell further comprises:
(i) one or more nucleic acid molecules encoding one or more enzymes of a tyramine containing hydroxycinnamic acid amide for making a hydroxycinnamoyl-CoA ester,
(ii) a nucleic acid molecule encoding a tyrosine decarboxylase (E.C. 4.1.1.25); and
(iii) a nucleic acid molecule encoding a tyramine N-hydroxycinnamoyltransferase (E.C. 2.3.1.110);
(b) cultivating the recombinant eukaryotic host cell for a time sufficient for the recombinant eukaryotic host cell to produce the tyramine containing hydroxycinnamic acid amide; and
(c) isolating the tyramine containing hydroxycinnamic acid amide from the recombinant eukaryotic host cell or from the cultivation supernatant, thereby producing a tyramine containing hydroxycinnamic acid amide.

9. The method of claim 8, wherein the one or more nucleic acid molecules encoding one or more enzymes of a phenylpropanoid CoA pathway for making a hydroxycinnamoylCoA ester comprising phenylalanine ammonia lyase, 4-coumarate-CoA ligase, cinnamate-4-hydroxylase, coumarate-3-hydroxylase, caffeoyl-CoA O-methyltransferase, 5-hydroxylase, caffeic acid/5-hydroxyferulic acid O-methyltransferase, tyrosine ammonia lyase, or a combination thereof.

10. The method of claim 8, wherein said recombinant eukaryotic host cell further overproduces S-adenosylmethionine.

11. The method of claim 8, wherein the recombinant eukaryotic host cell is a recombinant yeast strain.

12. The method of claim 11, wherein the yeast strain is selected from:

| Strain Name | Characteristics |
|---|---|
| Y0001 | aro10Δ pdc5Δ ARO4^{FBR} |
| | ARO7^{FBR} ARO1 TYDC |
| | PAL C4H CPR 4CL THT |
| Y0002 | aro10Δ pdc5Δ ARO4^{FBR} |
| | ARO7^{FBR} ARO1 TYDC |
| | PAL C4H CPR 4CL C3H |
| | THT |
| Y0003 | aro10Δ pdc5Δ ARO4^{FBR} |
| | ARO7^{FBR} ARO1 TYDC |
| | PAL C4H CPR 4CL C3H |
| | CCoAOMT THT |
| Y0004 | aro10Δ pdc5Δ ARO4^{FBR} |
| | ARO7^{FBR} ARO1 TYDC |
| | PAL C4H CPR 4CL C3H |
| | CCoA3H CCoAOMT F5H |
| | COMT THT |
| Y0005 | aro10Δ pdc5Δ ARO4^{FBR} |
| | ARO7^{FBR} ARO1 TYDC |
| | PAL 4CL THT |
| Y0006 | aro10Δ pdc5Δ ARO4^{FBR} |
| | ARO7^{FBR} ARO1 TYDC |
| | PAL C4H CPR 4CL THT |
| Y0007 | aro10Δ pdc5Δ ARO4^{FBR} |
| | ARO7^{FBR} ARO1 TYR1 |
| | TYDC 4CL THT |
| Y0008 | aro10Δ pdc5Δ ARO4^{FBR} |
| | ARO7^{FBR} ARO1 TYR1 |
| | TYDC 4CL THT |
| Y0009 | aro10Δ pdc5Δ ARO4^{FBR} |
| | ARO7^{FBR} ARO1 TYR1 |
| | TYDC 4CL THT |
| Y0010 | aro10Δ pdc5Δ ARO4^{FBR} |
| | ARO7^{FBR} ARO1 TYR1 |
| | TYDC 4CL THT |
| Y0011 | TYDC PAL C4H CPR |
| | 4CL THT |
| Y0012 | TYDC PAL C4H CPR |
| | 4CL C3H THT |
| Y0013 | TYDC PAL C4H CPR |
| | 4CL C3H CCoAMT THT |
| Y0014 | TYDC PAL C4H CPR |
| | 4CL C3H CCoA3H |
| | CCoAOMT F5H COMT |
| | THT |
| Y0015 | TYDC PAL 4CL THT |

13. The method of claim 8, wherein the tyramine containing hydroxycinnamic acid amide is selected from at least one of N-caffeoyltyramine, N-feruloyltyramine, 5-hydroxyferuloyltyramine, p-coumaroyltyramine, cinnamoyltyramine, sinapoyltyramine, (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, or 3-(3,4-dihydroxyphenyl)-N-(4-(methylsulfonamido)phenethyl)propanamide.

14. The method of claim 13, wherein the tyramine containing hydroxycinnamic acid amide is selected from at least one of N-caffeoyltyramine, N-feruloyltyramine, 5-hydroxyferuloyltyramine, p-coumaroyltyramine, cinnamoyltyramine, sinapoyltyramine.

15. An extract comprising a tyramine containing hydroxycinnamic acid amide produced by the method of claim 1.

16. A consumable product comprising the extract of claim 15.

17. The consumable product of claim 16, wherein the consumable product is a dietary supplement, food ingredient, additive, food product, a medical food, nutraceutical or pharmaceutical composition.

18. A method of cultivating a recombinant eukaryotic host cell, the recombinant eukaryotic host cell comprising an one or more nucleic acid molecules capable of producing a tyramine containing hydroxycinnamic acid amide or a tyramine containing hydroxycinnamic acid derivative, the method comprising the steps of:
a) inoculating the recombinant eukaryotic host cell into a growth medium,
b) cultivating the host cell to a desired cell density; and
c) harvesting the host cell from the culture medium.

19. The method of claim 18, wherein the recombinant eukaryotic host cell is a recombinant yeast strain selected from:

| Strain Name | Characteristics |
|---|---|
| Y0001 | aro10Δ pdc5Δ ARO4^{FBR} |
| | ARO7^{FBR} ARO1 TYDC |
| | PAL C4H CPR 4CL THT |
| Y0002 | aro10Δ pdc5Δ ARO4^{FBR} |
| | ARO7^{FBR} ARO1 TYDC |
| | PAL C4H CPR 4CL C3H |
| | THT |
| Y0003 | aro10Δ pdc5Δ ARO4^{FBR} |
| | ARO7^{FBR} ARO1 TYDC |
| | PAL C4H CPR 4CL C3H |
| | CCoAOMT THT |
| Y0004 | aro10Δ pdc5Δ ARO4^{FBR} |
| | ARO7^{FBR} ARO1 TYDC |
| | PAL C4H CPR 4CL C3H |
| | CCoA3H CCoAOMT F5H |
| | COMT THT |
| Y0005 | aro10Δ pdc5Δ ARO4^{FBR} |
| | ARO7^{FBR} ARO1 TYDC |
| | PAL 4CL THT |
| Y0006 | aro10Δ pdc5Δ ARO4^{FBR} |
| | ARO7^{FBR} ARO1 TYDC |
| | PAL C4H CPR 4CL THT |
| Y0007 | aro10Δ pdc5Δ ARO4^{FBR} |
| | ARO7^{FBR} ARO1 TYR1 |
| | TYDC 4CL THT |
| Y0008 | aro10Δ pdc5Δ ARO4^{FBR} |
| | ARO7^{FBR} ARO1 TYR1 |
| | TYDC 4CL THT |
| Y0009 | aro10Δ pdc5Δ ARO4^{FBR} |
| | ARO7^{FBR} ARO1 TYR1 |
| | TYDC 4CL THT |
| Y0010 | aro10Δ pdc5Δ ARO4^{FBR} |
| | ARO7^{FBR} ARO1 TYR1 |
| | TYDC 4CL THT |
| Y0011 | TYDC PAL C4H CPR |
| | 4CL THT |
| Y0012 | TYDC PAL C4H CPR |
| | 4CL C3H THT |
| Y0013 | TYDC PAL C4H CPR |
| | 4CL C3H CCoAMT THT |
| Y0014 | TYDC PAL C4H CPR |
| | 4CL C3H CCoA3H |
| | CCoAOMT F5H COMT |
| | THT |
| Y0015 | TYDC PAL 4CL THT |

20. The method of claim 18, wherein the tyramine containing hydroxycinnamic acid amide produced is selected from at least one of N-caffeoyltyramine, N-feruloyltyramine, 5-hydroxyferuloyltyramine, p-coumaroyltyramine, cinnamoyltyramine, sinapoyltyramine.
